(19) United States
(12) Patent Application Publication
(10) Pub. No.: US 2019/0142253 A1
(43) Pub. Date: May 16, 2019

(54) IMAGE PROCESSING DEVICE, ENDOSCOPE SYSTEM, INFORMATION STORAGE DEVICE, AND IMAGE PROCESSING METHOD

(71) Applicant: OLYMPUS CORPORATION, Tokyo (JP)

(72) Inventor: Jumpei TAKAHASHI, Tokyo (JP)

(73) Assignee: OLYMPUS CORPORATION, Tokyo (JP)

(21) Appl. No.: 16/227,093

(22) Filed: Dec. 20, 2018

Related U.S. Application Data
(63) Continuation of application No. PCT/JP2016/071159, filed on Jul. 19, 2016.

(51) Int. Cl. A61B 1/04; A61B 1/00; A61B 1/06

(52) U.S. Cl. CPC A61B 1/04; A61B 1/00009; A61B 1/07; A61B 1/0676; A61B 1/00186

(57) ABSTRACT

An image processing device comprising a processor including hardware, the processor performing: an image acquisition process of acquiring an image in time series; and a motion vector detection process including obtaining luminance identification information based on a pixel value of the image, and detecting a motion vector based on the image and the luminance identification information, the processor setting contribution in the motion vector detection process to be higher with a smaller luminance identified by the luminance identification information, the contribution being contribution of a low-frequency component of the image relative to a high-frequency component of the image.

### CROSS REFERENCE TO RELATED APPLICATION

This application is a continuation of International Patent Application No. PCT/JP2016/071159, having an international filing date of Jul. 19, 2016, which designated the United States, the entirety of which is incorporated herein by reference.

### BACKGROUND

Conventionally, methods (methods for detecting a motion vector) for positioning between frames have been widely known. Methods such as block matching have been widely used for detecting a motion vector. Noise reduction (hereinafter, also referred to as NR) between frames is implemented by taking a weighted average of a plurality of frames that have been positioned (have had position shift corrected) by using the motion vector detected. Thus, NR can be achieved while maintaining the sense of resolution.

The motion vector can be used in various processes other than NR.

Generally, a motion detection process, such as a block matching process, involves a risk of erroneous detection of a motion vector due to an influence of a noise component. When the motion vector erroneously detected is used to perform the NR process between frames, the sense of resolution is compromised and an image (artifact) which does not actually exist is generated.

In view of this, for example, JP-A-2006-23812 discloses a method of detecting a motion vector based on a frame that has been subjected to an NR process, so that an influence of noise can be reduced. An example of this NR process is a Low Pass Filter (LPF) process.

### SUMMARY

In accordance with one of some embodiments, there is provided an image processing device comprising a processor including hardware,

the processor performing:

an image acquisition process of acquiring an image in time series; and

a motion vector detection process including obtaining luminance identification information based on a pixel value of the image, and detecting a motion vector based on the image and the luminance identification information,

the processor

setting contribution in the motion vector detection process to be higher with a smaller luminance identified by the luminance identification information, the contribution being contribution of a low-frequency component of the image relative to a high-frequency component of the image.

In accordance with one of some embodiments, there is provided an endoscope system comprising: an imaging device that acquires an image in time series; and

a processor including hardware,

the processor performing:

a motion vector detection process including obtaining luminance identification information based on a pixel value of the image, and detecting a motion vector based on the image and the luminance identification information,

the processor setting contribution in the motion vector detection process to be higher with a smaller luminance identified by the luminance identification information, the contribution being contribution of a low-frequency component of the image relative to a high-frequency component of the image.

In accordance with one of some embodiments, there is provided an information storage device storing a program,

the program causing a computer to perform the steps of

acquiring an image in time series,

obtaining luminance identification information based on a pixel value of the image, and

detecting a motion vector based on the image and the luminance identification information,

the detecting of the motion vector including

setting contribution in the motion vector detection process to be higher with a smaller luminance identified by the luminance identification information, the contribution being contribution of a low-frequency component of the image relative to a high-frequency component of the image.

In accordance with one of some embodiments, there is provided an image processing method comprising: acquiring an image in time series;

obtaining luminance identification information based on a pixel value of the image; and

detecting a motion vector based on the image and the luminance identification information,

the detecting of the motion vector including

setting contribution in the motion vector detection process to be higher with a smaller luminance identified by the luminance identification information, the contribution being contribution of a low-frequency component of the image relative to a high-frequency component of the image.

### BRIEF DESCRIPTION OF THE DRAWINGS

FIG. 1 illustrates a configuration example of an endoscope system.

FIG. 2 illustrates a configuration example of an image sensor.

FIG. 3 illustrates an example of spectral characteristics of the image sensor.

FIG. 4 illustrates a configuration example of a motion vector detection section according to a first embodiment.

FIG. 5 includes FIG. 5A and FIG. 5B that each illustrate relationship between a subtraction ratio and a luminance signal.

FIG. 6 illustrates a setting example of an offset for correcting an evaluation value.

FIG. 7 is a diagram illustrating relationship between a coefficient for correcting the evaluation value and the luminance signal.

FIG. 8 illustrates an example of prediction information for obtaining information about noise from an image.

FIG. 9 is a flowchart illustrating a process according to some embodiments.

FIG. 10 illustrates a configuration example of a motion vector detection section according to a second embodiment.

FIG. 11 includes FIG. 11A to FIG. 11C illustrating an example of a plurality of filter with different smoothing levels.

FIG. 12 illustrates a configuration example of a motion vector detection section according to a third embodiment.

### DESCRIPTION OF EXEMPLARY EMBODIMENTS

The following disclosure provides many different embodiments, or examples, for implementing different features of the provided subject matter. These are, of course, merely examples and are not intended to be limiting. In addition, the present disclosure may repeat reference numerals and/or letters in the various examples. This repetition is for the purpose of simplicity and clarity and does not in itself dictate a relationship between the various embodiments and/or configurations discussed. Further, when a first element is described as being “connected” or “coupled” to a second element, such description includes embodiments in which the first and second elements are directly connected or coupled to each other, and also includes embodiments in which the first and second elements are indirectly connected of coupled to each other with one or more other intervening elements in between.

In accordance with one of some embodiments, there is provided an image processing device comprising a processor including hardware,

the processor performing:

an image acquisition process of acquiring an image in time series; and

a motion vector detection process including obtaining luminance identification information based on a pixel value of the image, and detecting a motion vector based on the image and the luminance identification information,

the processor

setting contribution in the motion vector detection process to be higher with a smaller luminance identified by the luminance identification information, the contribution being contribution of a low-frequency component of the image relative to a high-frequency component of the image.

In accordance with one of some embodiments, there is provided an endoscope system comprising: an imaging device that acquires an image in time series; and

a processor including hardware,

the processor performing:

a motion vector detection process including obtaining luminance identification information based on a pixel value of the image, and detecting a motion vector based on the image and the luminance identification information,

the processor setting contribution in the motion vector detection process to be higher with a smaller luminance identified by the luminance identification information, the contribution being contribution of a low-frequency component of the image relative to a high-frequency component of the image.

In accordance with one of some embodiments, there is provided an information storage device storing a program,

the program causing a computer to perform the steps of

acquiring an image in time series,

obtaining luminance identification information based on a pixel value of the image, and

detecting a motion vector based on the image and the luminance identification information,

the detecting of the motion vector including

setting contribution in the motion vector detection process to be higher with a smaller luminance identified by the luminance identification information, the contribution being contribution of a low-frequency component of the image relative to a high-frequency component of the image.

In accordance with one of some embodiments, there is provided an image processing method comprising: acquiring an image in time series;

obtaining luminance identification information based on a pixel value of the image; and

detecting a motion vector based on the image and the luminance identification information,

the detecting of the motion vector including

setting contribution in the motion vector detection process to be higher with a smaller luminance identified by the luminance identification information, the contribution being contribution of a low-frequency component of the image relative to a high-frequency component of the image.

Embodiments will now be described. The embodiments described below are not intended to unduly limit the present disclosure set forth in the claims. Not all the components described in the embodiments are essential components of the present disclosure.

While first to third embodiments below mainly describe examples of endoscope systems, methods according to the embodiments are applicable to image processing devices, not limited to endoscope systems. Examples of the image processing devices may include general-use equipment such as personal computers (PCs) and server systems and special-purpose equipment such as application specific integrated circuits (ASICs) and custom ICs. Images to be processed by the image processing devices may include, but not limited to, images (in-vivo images) captured by an imaging device in an endoscope system, and various types of images can be processed by the image processing devices.

### 1. First Embodiment

### 1.1 System Configuration Example

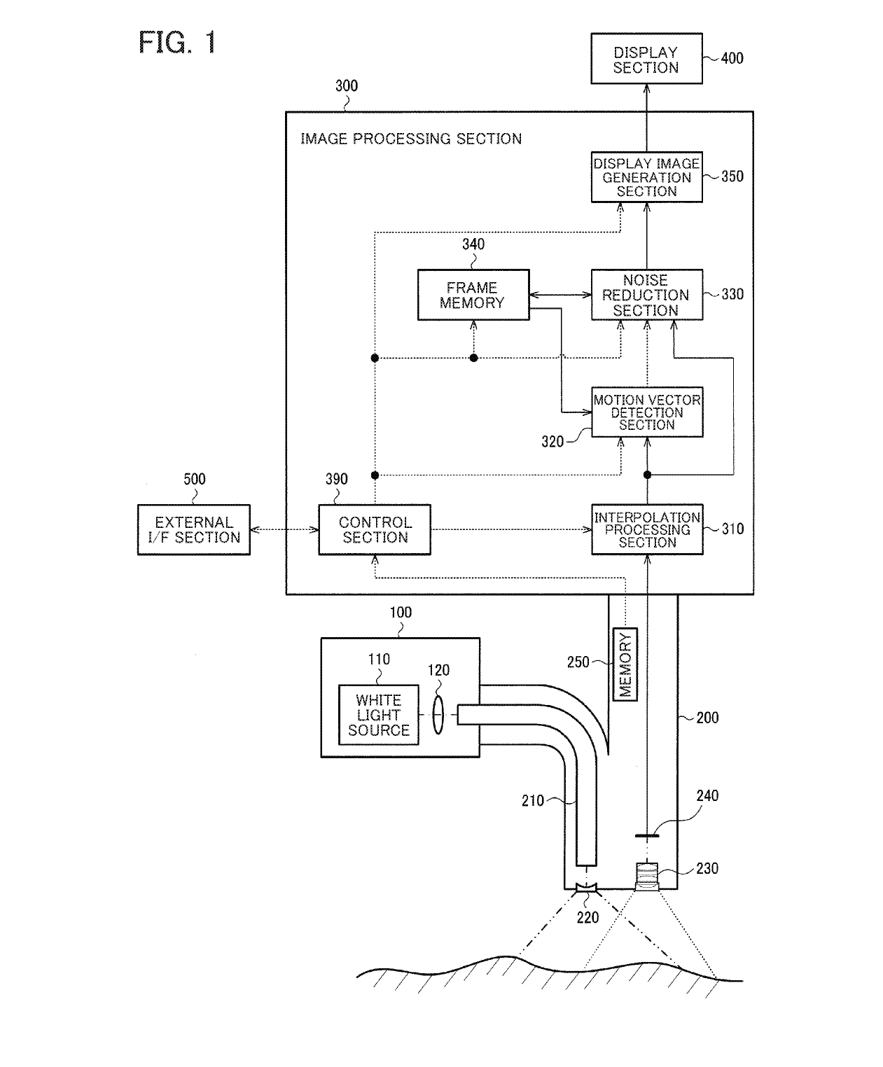

An endoscope system according to a first embodiment of the present disclosure is described with reference to FIG. 1. The endoscope system according to the present embodiment includes a light source section 100, an imaging device 200, an image processing section 300, a display section 400, and an external I/F section 500.

The light source section 100 includes a white light source 110 that generates white light and a lens 120 that condenses the white light into a light guide fiber 210.

The imaging device 200 is formed to be in an elongated shape and can be curved so as to be capable of being inserted into a body cavity. The imaging device has a detachably attached structure so that different imaging devices can be used for different monitored portions. The imaging device 200 is hereinafter also referred to as a scope.

The imaging device 200 includes the light guide fiber 210 that guides the light condensed by the light source section 100, an illumination lens 220 that diffuses the light guided by the light guide fiber 210 so that an object is irradiated with the resultant light, a condensing lens 230 that condenses reflected light from the object, an image sensor 240 that detects the reflected light condensed by the condensing lens 230, and a memory 250. The memory 250 is connected to a control section 390 described later.

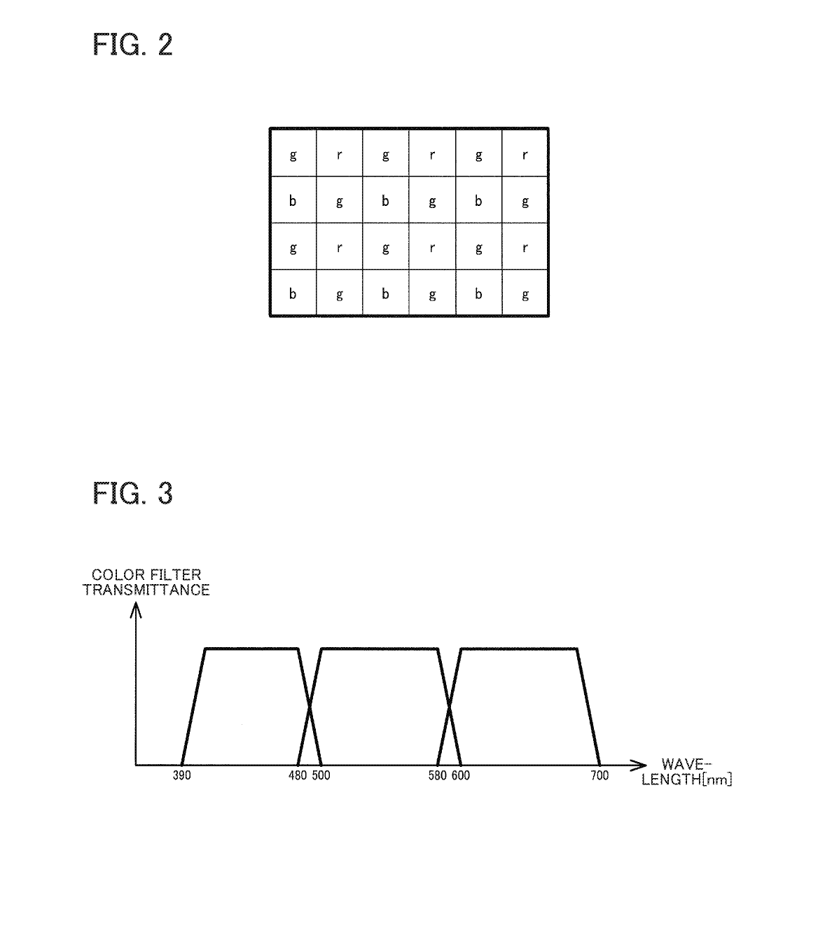

The image sensor 240 is an image sensor having a Bayer array as illustrated in FIG. 2. FIG. 2 illustrates color filters r, g, and b that correspond to three colors and are characterized in that the r filter transmits light with a wavelength in a range from 580 to 700 nm, the g filter transmits light with a wavelength in a range from 480 to 600 nm, and the b filter transmits light with a wavelength in a range from 390 to 500 nm as illustrated in FIG. 3.

The memory 250 holds an ID number unique to each scope. Thus, the control section 390 can refer to the ID number held by the memory 250 to identify the type of the scope connected.

The image processing section 300 includes an interpolation processing section 310, a motion vector detection section 320, a noise reduction section 330, a frame memory 340, a display image generation section 350, and a control section 390.

The interpolation processing section 310 is connected to the motion vector detection section 320 and the noise reduction section 330. The motion vector detection section 320 is connected to the noise reduction section 330. The noise reduction section 330 is connected to the display image generation section 350. The frame memory 340 is connected to the motion vector detection section 320, and is also bidirectionally connected with the noise reduction section 330. The display image generation section 350 is connected to the display section 400. The control section 390 is connected to and controls the interpolation processing section 310, the motion vector detection section 320, the noise reduction section 330, the frame memory 340, and the display image generation section 350.

The interpolation processing section 310 performs an interpolation process on an image acquired by the image sensor 240. As described above, the image sensor 240 has the Bayer array illustrated in FIG. 2, and thus each pixel of the image acquired by the image sensor 240 only has one of R, G, and B signal values, and thus lacks the remaining two of the R, G, and B signal values.

Thus, the interpolation processing section 310 performs the interpolation process on each pixel of the image to interpolate the lacking signal values, whereby an image with each pixel having all of the R, G, and B signal values is generated. For example, a known bicubic interpolation process may be performed as the interpolation process. Here, the image generated by the interpolation processing section 310 will be referred to as an RGB image. The interpolation processing section 310 outputs the RGB image thus generated to the motion vector detection section 320 and the noise reduction section 330.

The motion vector detection section 320 detects a motion vector (Vx(x,y),Vy(x,y)) for each pixel of the RGB image. Here, an x axis represents a horizontal direction (left and right direction) of the image, and a y axis represents a vertical direction (upper and lower direction), and (x,y) that is a set of an x coordinate value and a y coordinate value represents a pixel in the image. The motion vector (Vx(x,y),Vy(x,y)) includes Vx(x,y) representing a motion vector components in the x (horizontal) direction at the pixel (x,y), and Vy(x,y) representing a motion vector components in the y (vertical) direction at the pixel (x,y). The origin (0,0) is assumed to be at an upper left corner of the image.

The motion vector is detected by using an RGB image at a process target timing (an RGB image acquired at a latest timing in a narrow sense) and a recursive RGB image stored in the frame memory 340. The recursive RGB image is an RGB image after the noise reduction process acquired at a timing before the RGB image at the process target timing, and is an image as a result of performing the noise reduction process on an RGB image acquired at a preceding timing (preceding frame) in a narrow sense. In this specification, the RGB image at the process target timing is hereinafter simply referred to as an “RGB image”.

A method of detecting a motion vector is based on a known block matching. The block matching searches a target image (recursive RGB image) for a position of a block with high correlation relative to a certain block in a reference image (RGB image). A relative shifted amount between these blocks corresponds to a motion vector of the certain block. A value for identifying the correlation between the blocks is defined as an evaluation value. A lower evaluation value indicates higher correlation between blocks. The process performed by the motion vector detection section 320 is described in detail later.

The noise reduction section 330 uses an RGB image output from the interpolation processing section 310 and a recursive RGB image output from the frame memory 340, to perform the NR process on the RGB image. Specifically, a G component G_{NR}(x,y) at coordinates (x,y) in the image after the NR process (hereinafter, referred to as an NR image) may be obtained by the following Formula (1). In the following Formula (1), G_{cur}(x,y) represents a pixel value of a G component at coordinates (x,y) in the RGB image and Gₚᵣₑ(x,y) represents a pixel value of a G component at coordinates (x,y) in the recursive RGB image.

G_{NR}(x,y)=we_cur×G_{cur}(x,y)+(1−we_cur)×Gₚᵣₑ{x+Vx(x,y),y+Vy(x,y)} (1),

where we_cur is a value satisfying 0<we_cur≤1. A smaller value indicates a higher rate of a pixel value acquired at a past timing, and thus involves higher recursion amount and a higher noise reduction level. For we_cur, a predetermined value may be set in advance or a desired value may be set by a user through the external I/F section 500. The process that is the same as that for the G signal described above is also performed on the R and the B signals.

The noise reduction section 330 outputs an NR image to the frame memory 340. The frame memory 340 holds the NR image. The NR image is used as the recursive RGB image in a process for the RGB image subsequently acquired.

The display image generation section 350 performs a known process, such as white balance and a color or gradation conversion process, on the NR image output from the noise reduction section 330 to generate a display image. The display image generation section 350 outputs the display image thus generated to the display section 400. An example of the display section 400 includes a display device such as a liquid crystal display device.

The external I/F section 500 is an interface with which a user performs an operation such as input on the endoscope system (image processing device), and includes a power switch for turning ON/OFF the power, a mode switching button for switching among an image capturing mode and other various modes, and the like. The external I/F section 500 outputs information input thereto to the control section 390.

### 1.2 Detail of Motion Vector Detection Process

In an endoscopic image, a block with high correlation is searched for based on a biological structure (a blood vessel and a duct). In this process, a block is preferably searched for based on information about a fine biological structure (such as capillary blood vessel) distributed in a mid to high frequency band in the image, so that a highly accurate motion vector can be detected. However, a large amount of noise would hide the fine biological structure, and thus the motion vector is detected with a lower accuracy and a higher risk of erroneous detection. When a noise reduction process (lowpass filter (LPF) process) is uniformly performed as in JP-A-2006-23812, an area where the fine biological structure is remaining due to a small amount of noise also becomes the target of the process, resulting in the fine biological structure blurred. As a result, the detection accuracy is compromised in the area where the motion vector would have been detectable highly accurately.

In view of this, the present embodiment features control on a method of calculating an evaluation value based on a brightness of the image. Thus, the motion vector can be highly accurately detected in a bright portion with a small amount of noise, while preventing the erroneous detection in a dark portion with a large amount of noise.

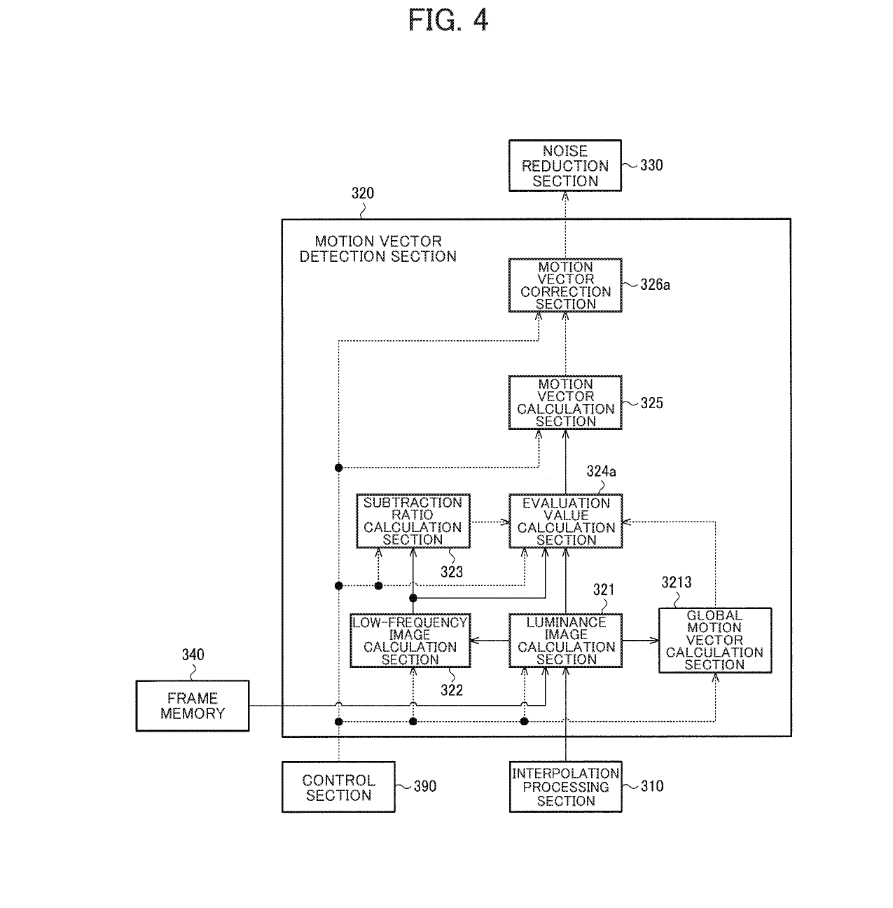

The motion vector detection section 320 is described in detail. As illustrated in FIG. 4, the motion vector detection section 320 includes a luminance image calculation section 321, a low-frequency image calculation section 322, a subtraction ratio calculation section 323, an evaluation value calculation section 324a, a motion vector calculation section 325, a motion vector correction section 326a, and a global motion vector calculation section 3213.

The interpolation processing section 310 and the frame memory 340 are connected to the luminance image calculation section 321. The luminance image calculation section 321 is connected to the low-frequency image calculation section 322, the evaluation value calculation section 324a, and the global motion vector calculation section 3213. The low-frequency image calculation section 322 is connected to the subtraction ratio calculation section 323. The subtraction ratio calculation section 323 is connected to the evaluation value calculation section 324a. The evaluation value calculation section 324a is connected to the motion vector calculation section 325. The motion vector calculation section 325 is connected to the motion vector correction section 326a. The motion vector correction section 326a is connected to the noise reduction section 330. The global motion vector calculation section 3213 is connected to the evaluation value calculation section 324a. The control section 390 is connected to and controls the components of the motion vector detection section 320.

The luminance image calculation section 321 calculates a luminance image from the RGB image output from the interpolation processing section 310 and the recursive RGB image output from the frame memory 340. Specifically, the luminance image calculation section 321 calculates a Y image from the RGB image, and calculates a recursive Y image from the recursive RGB image. Specifically, a pixel value Y_{cur} of the Y image and a pixel value Yₚᵣₑ of the recursive Y image may be obtained with the following Formula (2). Y_{cur}(x,y) represents a signal value (luminance value) at coordinates (x,y) in the Y image, and Yₚᵣₑ(x,y) represents a signal value at coordinates (x,y) in the recursive Y image. The same applies to pixel values in R, G, and B images. The luminance image calculation section 321 outputs the Y image and the recursive Y image to the low-frequency image calculation section 322, the evaluation value calculation section 324a, and the global motion vector calculation section 3213.

Y_{cur}(x,y)={R_{cur}(x,y)+2×G_{cur}(x,y)+B_{cur}(x,y)}/4

Yₚᵣₑ(x,y)={Rₚᵣₑ(x,y)+2×Gₚᵣₑ(x,y)+Bₚᵣₑ(x,y)}/4 (2)

For example, the global motion vector calculation section 3213 calculates a global motion vector (Gx,Gy) indicating a shifted amount over the entire image between the reference image and the target image, through the block matching described above, and outputs the global motion vector (Gx,Gy) to the evaluation value calculation section 324a. The global motion vector may be calculated with a larger kernel size (block size) in the block matching than in a case of obtaining a local motion vector (a motion vector output from the motion vector detection section 320 in the present embodiment). For example, the global motion vector may be calculated with the kernel size in the block matching set to be the size of the image itself. The global motion vector is calculated through the block matching over the entire image, and thus is less susceptible to noise.

The low-frequency image calculation section 322 performs a smoothing process on the Y image and the recursive Y image, to calculate a low-frequency image (a low-frequency Y image and a recursive low-frequency Y image). Specifically, a pixel value Y_LPF_{cur} of the low-frequency Y image and a pixel value Y_LPFₚᵣₑ of the low-frequency recursive Y image may be obtained with the following Formula (3). The low-frequency image calculation section 322 outputs the low-frequency Y image to the subtraction ratio calculation section 323, and outputs the low-frequency Y image and the recursive low-frequency Y image to the evaluation value calculation section 324a.

$$\begin{matrix}Formula 1 & \\ {Y\_LPF}_{cur} ( x , y ) = \frac{{\sum }_{i = - 1}^{1} {\sum }_{j = - 1}^{1} {Y}_{cur} ( x + i , y + j )}{9} & \\ {Y\_LPF}_{pre} ( x , y ) = \frac{{\sum }_{i = - 1}^{1} {\sum }_{j = - 1}^{1} {Y}_{pre} ( x + i , y + j )}{9} & ( 3 )\end{matrix}$$

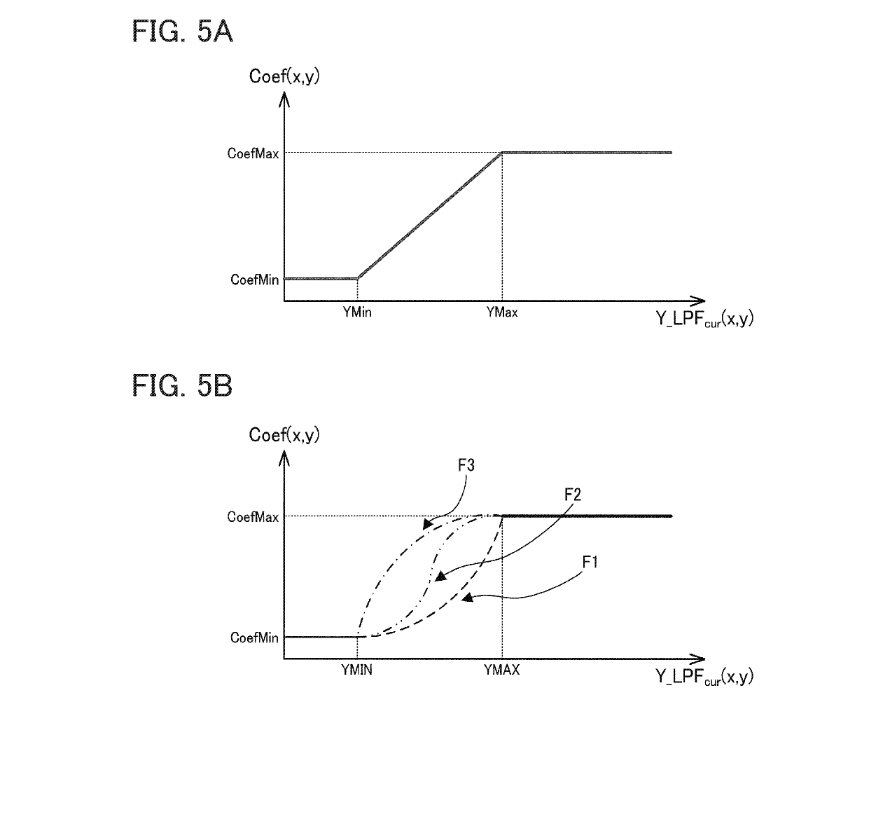

The subtraction ratio calculation section 323 calculates a subtraction ratio Coef(x,y) for each pixel through the following Formula (4) based on the low-frequency Y image. CoefMin represents the minimum value of the subtraction ratio Coef and CoefMax represents the maximum value of the subtraction ratio Coef(x,y), and these values satisfy the relationship 1≥CoefMax>CoefMin≥0. Ymin represents a given lower luminance threshold and Ymax represents a given upper luminance threshold. When each pixel is allocated with 8-bit information, the luminance value is a value equal to or larger than 0 and equal to or lower than 255, and thus Ymin and Ymax satisfy the relationship 255≥YMax>YMin≥0. FIG. 5A illustrates a characteristic of the subtraction ratio Coef(x,y).

$$\begin{matrix}Formula 2 & \\ Coef ( x , y ) = \{ \begin{matrix}if ( {Y\_LPF}_{cur} ( x , y ) \leq Y Min ) & Coef Min \\ elseif ( {Y\_LPF}_{cur} ( x , y ) \geq Y Max ) & Coef Max \\ else & Coef Min + ( Coef Max - Coef Min ) \frac{{Y\_LPF}_{cur} ( x , y ) - Y Min}{Y Max - Y Min}\end{matrix} & ( 4 )\end{matrix}$$

As can be seen in Formula (4) and FIG. 5A, the subtraction ratio Coef(x,y) is a coefficient increasing and decreasing as the pixel value (luminance value) of the low-frequency Y image increases and decreases. However, the characteristic of the subtraction ratio Coef(x,y) is not limited to this. Any characteristic may be employed as long as the subtraction ratio Coef(x,y) increases in accordance with Y_LPF_{cur}(x,y). FIG. 5B illustrates exemplary characteristics F1 to F3 that can be employed.

The evaluation value calculation section 324a calculates an evaluation value SAD(x+m+Gx,y+n+Gy) based on the following Formula (5). A mask in the following Formula (5) represents the kernel size of the block matching. As can be seen in the following Formula (5), variables p and q each vary within a range of − mask to + mask, and thus the kernel size is 2×mask+1.

$$\begin{matrix}Formula 3 & \\ SAD ( x + m + Gx , y + n + Gy ) = \{ {\sum }_{p = - mask}^{mask} {\sum }_{q = - mask}^{mask} \vert \begin{matrix}{Y}_{cur}^{′} ( x + p , y + q ) - \\ {Y}_{pre}^{′} ( x + p + m + Gx , y + q + n + Gy )\end{matrix} \vert \} + {Coef}^{′} ( x , y ) \times offset ( m , n ) \\ {Y}_{cur}^{′} ( x , y ) = {Y}_{cur} ( x , y ) - {Y\_LPF}_{cur} ( x , y ) \times Coef ( x , y ) \\ {Y}_{pre}^{′} ( x , y ) = {Y}_{pre} ( x , y ) - {Y\_LPF}_{pre} ( x , y ) \times Coef ( x , y ) & ( 5 )\end{matrix}$$

In the formula, m+Gx,n+Gy represents the relative shifted amount between the reference image and the target image, m represents a motion vector search range in the x direction, and n represents a motion vector search range in the y direction. For example, m and n are each an integer value between −2 and +2. Thus, a plurality of (5×5=25) the evaluation values are calculated based on Formula (5) described above.

In the present embodiment, the evaluation value is calculated based on the global motion vector (Gx,Gy). Specifically, the motion vector detection is performed with a search range defined by m and n based on the global motion vector set as a target, as can be seen in Formula (5) described above. Note that this search range may not be used. The range defined by m and n (the motion vector search range), which is ±2 pixels in the above description, may be set by a user through the external I/F section 500 to be a desired value. The mask corresponding to the kernel size may be of a predetermined value or may be set by the user through the external I/F section 500. Similarly, CoefMax, CoefMin, YMax, and YMin may be set to be a predetermined value in advance or may be set by the user through the external I/F section 500.

As can be seen in the first term in Formula (5) described above, an image (motion detection image) to be the target of the evaluation value calculation in the present embodiment is obtained by subtracting a low-frequency image from a luminance image, based on the subtraction ratio Coef(x,y) (a coefficient of the low-frequency luminance image). The subtraction ratio Coef(x,y) decreases as the luminance decreases as illustrated in FIG. 5A. Thus, a smaller luminance results in more low-frequency components remaining and a larger luminance results in more low-frequency component subtracted. Thus, a process with relatively more weight on low-frequency components is performed when the luminance is small, and a process with relatively more weight on high-frequency components is performed when the luminance is large.

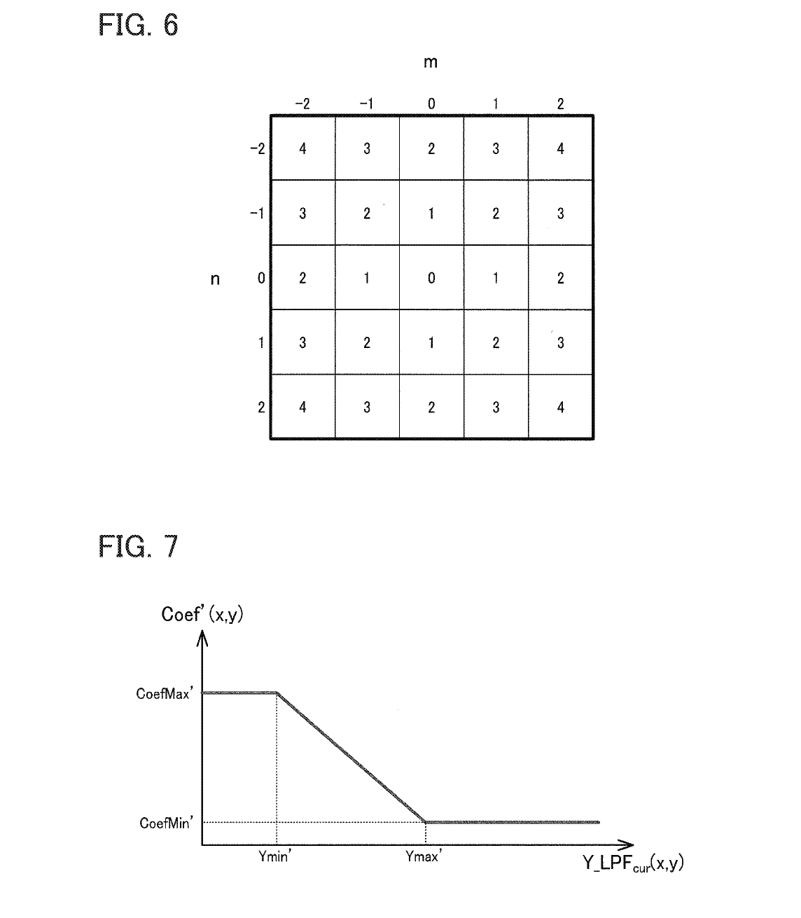

The evaluation value according to the present embodiment is a result of performing correction in the second term on the first term for obtaining a sum of absolute differences. Offset(m,n) in the second term is a correction value according to the shifted amount. FIG. 6 illustrates specific values of Offset(m,n). The correction value is not limited to those in FIG. 6, and may be any value that becomes larger at a portion farther from the search origin (m,n)=(0,0).

A coefficient Coef′(x,y) is determined based on Y_LPF_{cur}(x,y), as in the case of Coef(x,y). FIG. 7 illustrates a characteristic of Coef′(x,y). Note that Coef′(x,y) is not limited to this characteristic and may have any characteristic of decreasing as Y_LPF_{cur}(x,y) increases. Variables in FIG. 7 satisfy the relationship CoefMax′>CoefMin′≥0 and the relationship 255>YMax′≥YMin′≥0. CoefMax′, CoefMin′, YMax′, and YMin′ may be set to be a predetermined value in advance, or may be set by the user through the external I/F section 500.

As illustrated in FIG. 7, Coef′(x,y) decreases as Y_LPF_{cur}(x,y) increases. Thus, a small Y_LPF_{cur}(x,y), corresponding to the dark portion, leads to a large value of Coef′(x,y), resulting in a high contribution of the second term to the evaluation value. As illustrated in FIG. 6, Offset(m,n) is characterized in that it is a larger value at a portion farther from the search origin. Thus, when the contribution of the second term is high, the evaluation value tends to be small at the search origin and to be larger at a portion farther from the search origin. With the second term used for calculating the evaluation value, a vector corresponding to the search origin, that is, the global motion vector (Gx,Gy) is likely to be selected as the motion vector in a dark portion.

The motion vector calculation section 325 detects a shifted amount (m_min,n_min) corresponding to the minimum evaluation value SAD(x+m+Gx,y+n+Gy) as the motion vector (Vx′(x,y),Vy′(x,y)) as illustrated in the following Formula (6). In the formula, m_min represents a sum of m corresponding to the minimum evaluation value and an x component Gx of the global motion vector, and n_min represents a value of n corresponding to the minimum evaluation value and a y component Gy of the global motion vector.

Vx′(x,y)=m_min

Vy′(x,y)=n_min (6)

The motion vector correction section 326a multiplies the motion vector (Vx′(x,y),Vy′(x,y)), calculated by the motion vector calculation section 325, by the correction coefficient C(0<C<1) to obtain the motion vector (Vx(x,y),Vy(x,y)) to be output from the motion vector detection section 320. The correction coefficient C is characterized in that it increases in accordance with the Y_LPF_{cur}(x,y), as in the case of Coef(x,y) illustrated in FIG. 5A and FIG. 5B. When the luminance is equal to or smaller than a predetermined value, the correction coefficient C may be set to be zero, so that the motion vector is forcibly set to be the global motion vector (Gx,Gy). The following Formula (7) defines the correction process performed by the motion vector correction section 326a.

Vx(x,y)=C×{Vx′(x,y)−Gx}+Gx

Vy(x,y)=C×{Vy′(x,y)−Gy}+Gy (7)

As described above with an example of the endoscope system, an image processing device according to the present embodiment includes an image acquisition section that acquires images in time series, and the motion vector detection section 320 that obtains luminance identification information based on a pixel value of the image and detects a motion vector based on the image and the luminance identification information. The motion vector detection section 320 sets a contribution of a low-frequency component of the image relative to a high-frequency component of the image in a detection process for the motion vector to be higher with a smaller luminance identified by the luminance identification information.

The image processing device according to the present embodiment may have a configuration corresponding to the image processing section 300 in the endoscope system illustrated in FIG. 1. In such a case, the image acquisition section may be implemented as an interface that acquires an image signal from the imaging device 200, and may be an A/D converter that performs A/D conversion on an analog signal from the imaging device 200 for example.

Alternatively, the image processing device may be an information processing device that acquires image data including images in time series from an external device, and performs a detection process for a motion vector with the image data as a target. In such a case, the image acquisition section may be implemented as an interface for an external device, and may be a communication section (more specifically, hardware such as a communication antenna) that communicates with the external device.

Alternatively, the image processing device itself may include an imaging device that captures an image. In such a case, the image acquisition section is implemented by the imaging device.

The luminance identification information according to the present embodiment is information with which luminance and brightness of an image can be identified, and is a luminance signal in a narrow sense. The luminance signal may be the pixel value Y_LPF_{cur}(x,y) of the low-frequency Y image as described above, or may be a pixel value Y_{cur}(x,y) of the Y image as described later in a second embodiment. The luminance identification information may be another kind of information as in a modification described later.

With the method according to the present embodiment, a spatial frequency band, used for detecting a motion vector, can be controlled in accordance with a luminance of an image. Thus, in a bright portion with a small amount of noise, a motion vector can be detected with high accuracy based on information (fine capillary blood vessels and the like) about a mid to high frequency band of an RGB image. Furthermore, in a dark portion with a large amount of noise, a motion vector is detected based on information about a low frequency band (a thick blood vessel or a wall of a digestive tract), so that erroneous detection due to noise can be reduced from that in a case where information about a mid to high frequency band is used.

Specifically, as can be seen in Formulae (4) and (5) described above, a coefficient of determination of the low-frequency component in calculation for the evaluation value is controlled in accordance with the signal value Y_LPF_{cur}(x,y) of the low-frequency Y image indicating the brightness (luminance) of an RGB image. For a bright portion with a small amount of noise, Coef(x,y) is set to be large so that the coefficient of determination of the low-frequency component becomes small (the coefficient of determination of the high-frequency component becomes large). Thus, a motion vector can be detected highly accurately based on information about fine capillary blood vessels and the like. On the other hand, for a dark portion with a large amount of noise, Coef(x,y) is set to be small so that the coefficient of determination of the low-frequency component becomes large (the coefficient of determination of the high-frequency component becomes small). Thus, high resistance against noise can be achieved, whereby erroneous detection of the motion vector can be suppressed.

With the process described above, the motion vector can be detected highly accurately regardless of noise in an input image. With the noise reduction process as represented in Formula (1) and the like described above, the motion vector can be highly accurately detected in the bright portion so that noise can be reduced while maintaining a contrast of blood vessels and the like. Furthermore, the erroneous detection in the dark portion due to noise is suppressed, whereby an effect of suppressing a motion (artifact) that is not actually made by an object can be obtained.

The motion vector detection section 320 generates a motion detection image used for the motion vector detection process based on an image, and sets a rate of the low-frequency component in the motion detection image to be higher in a case where the luminance identified by the luminance identification information is small than in a case where the luminance is large.

The motion detection image is an image acquired based on the RGB image and the recursive RGB image, and is used for the motion vector detection process. More specifically, the motion detection image is an image used for an evaluation value calculation process and is Y′_{cur}(x,y) and Y′ₚᵣₑ(x,y) in Formula (5) described above.

More specifically, the motion vector detection section 320 generates a smoothed image (Y_LPF_{cur}(x,y) and Y_LPFₚᵣₑ(x,y) that are low-frequency images in the example described above) by performing a predetermined smoothing filter process on an image. Then, the motion vector detection section 320 generates the motion detection image by subtracting the smoothed image from the image at a first subtraction ratio in a case where the luminance identified by the luminance identification information is small, and generates the motion detection image by subtracting the smoothed image from the image at a second subtraction ratio higher than the first subtraction ratio in a case where the luminance identified by the luminance identification information is large.

As illustrated in FIG. 5A and FIG. 5B, the subtraction ratio Coef(x,y) is characterized in that it increases as the luminance increases. Thus, in the motion detection image, a smaller luminance leads to a smaller subtraction ratio of the low-frequency component, and thus results in a larger ratio of the low-frequency component than in a case where the luminance is large.

Thus, the motion vector can be appropriately detected in accordance with the luminance by controlling the frequency band of the motion detection image. Specifically, the frequency band of the motion detection image is controlled with the luminance controlled in accordance with the subtraction ratio Coef(x,y). When the subtraction ratio Coef(x,y) is used, the ratio of the low-frequency component in the motion detection image can be relatively freely changed. For example, as illustrated in FIG. 5A and FIG. 5B, if Coef(x,y) is characterized in that it continuously changes relative to the luminance, the ratio of the low-frequency component in the motion detection image obtained by using Coef(x,y) can also be changed continuously (with a finer unit) in accordance with the luminance.

In the second embodiment described later, the motion detection image is an image obtained by performing a filter process with any one of filters A to C, and thus the frequency band of the motion detection image is controlled by switching the filter coefficient itself. Thus, the method according to the second embodiment requires a large number of filters for controlling the ratio of the low-frequency component in the motion detection image in detail. Thus, the method might involve hardware disadvantages such as an increase in the number of filter circuits or an increase in process time due to time division use of the filter circuit, or might involve excessive consumption of a memory capacity due to storage of a large number of motion detection images (corresponding to the number of filters). Thus, the method according to the present embodiment is advantageous in that the circuit configuration is less likely to be complex or the memory capacity is less likely to be excessively consumed, compared with the second embodiment described later.

The motion vector detection section 320 (evaluation value calculation section 324a) calculates a difference between a plurality of images acquired in time series as an evaluation value, and detects a motion vector based on the evaluation value. The motion vector detection section sets the contribution of the low-frequency component of the image relative to the high-frequency component of the image in the calculation process for the evaluation value to be higher with a smaller luminance identified by the luminance identification information.

Thus, the relative contribution of the low-frequency component to the evaluation value is controlled so that the motion vector detection process can be appropriately implemented in accordance with the luminance. This is implemented with Y′_{cur}(x,y) and Y′ₚᵣₑ(x,y) used for calculation of the first term in Formula (5) described above.

The motion vector detection section 320 (evaluation value calculation section 324a) may correct the evaluation value to facilitate detection of a given reference vector. Specifically, the motion vector detection section 320 corrects the evaluation value so that the detection of the reference vector is further facilitated with a smaller luminance identified by the luminance identification information.

This reference vector may be the global motion vector (Gx,Gy) representing a global motion as compared with a motion vector detected based on the evaluation value as described above. The “motion vector detected based on an evaluation value” is a motion vector to be obtained with the method according to the present embodiment, and corresponds to (Vx(x,y),Vy(x,y)) or (Vx′(x,y),Vy′(x,y)). The global motion vector involves a kernel size in the block matching larger than that in a case of Formula (5) described above, and thus serves as information roughly representing a motion between images. Note that the reference vector is not limited to the global motion vector and may be a zero vector (0,0) for example.

The correction for the evaluation value to make the reference vector likely to be detected corresponds to the second term in Formula (5) described above. Thus, the correction can be implemented with Coef′(x,y) and Offset(m,n). When the luminance is small and thus the amount of noise is large, a local variation of a motion vector resulting in a value of (m,n), corresponding to the minimum evaluation value, different from (0,0) is likely to be caused by noise (local noise in particular), and thus the reliability of the value obtained is low. On the other hand, in the present embodiment, Coef′(x,y) in Formula (5) described above is set to be large for a dark portion so that the reference vector is likely to be selected, whereby variation of a motion vector due to noise can be suppressed.

The motion vector detection section 320 (motion vector correction section 326a) performs a correction process on the motion vector obtained based on the evaluation value. The motion vector detection section 320 may perform the correction process on the motion vector based on the luminance identification information so that the motion vector becomes close to the given reference vector. Specifically, the motion vector detection section 320 may perform the correction process so that the motion vector becomes closer to the given reference vector with a smaller luminance identified by the luminance identification information.

The “motion vector obtained based on the evaluation value” corresponds to (Vx′(x,y),Vy′(x,y)) in the example described above, and a motion vector after the correction process corresponds to (Vx(x,y),Vy(x,y)). Specifically, the correction process corresponds to Formula (7) described above.

Thus, the variation of the motion vector in the dark portion can be more effectively suppressed to achieve higher noise resistance, with a process different from correction on the evaluation value with Coef′(x,y) and Offset(m,n).

As described above with reference to FIG. 1 and the like, the method according to the present embodiment can be applied to an endoscope system including: the imaging device 200 that captures images in time series; and the motion vector detection section 320 that obtains the luminance identification information based on a pixel value of the image and detects a motion vector based on the image and the luminance identification information. The motion vector detection section 320 in the endoscope system sets contribution of a low-frequency component of the image relative to a high-frequency component of the image in the motion vector detection process to be higher with a smaller luminance identified by the luminance identification information.

The image processing section 300 according to the present embodiment has components implemented by hardware. However, the present disclosure is not limited to this, and may be implemented by software, with a configuration, such as a capsule endoscope for example, where a central processing unit (CPU) executes the processes of the components on an image acquired in advance by an image sensor. Alternatively, a part of the processes of the components may be implemented by software.

Thus, the method according to the present embodiment can be applied to a program that causes a computer to perform the steps of acquiring an image in time series, obtaining luminance identification information based on a pixel value of the image, and detecting a motion vector based on the image and the luminance identification information, the detecting of the motion vector including setting contribution of a low-frequency component of the image relative to a high-frequency component of the image in the motion vector detection process to be higher with a smaller luminance identified by the luminance identification information.

In such a case, the image processing device according to the present embodiment and the like are implemented with a processor such as a CPU executing a program. Specifically, a program stored in a non-transitory information storage device is read and executed by the processor such as a CPU. The information storage device (computer readable device) stores a program and data. A function of the information storage device can be implemented with an optical disk (such as a digital versatile disk or a compact disk), a hard disk drive (HDD), or a memory (such as a card-type memory or a read only memory (ROM)). The processor such as a CPU performs various processes according to the present embodiment based on a program (data) stored in the information storage device. Thus, the information storage device stores a program (a program causing a computer to execute the processes of the components) causing a computer (a device including an operation element, a processor, a storage, and an output element) to function as components according to the present embodiment.

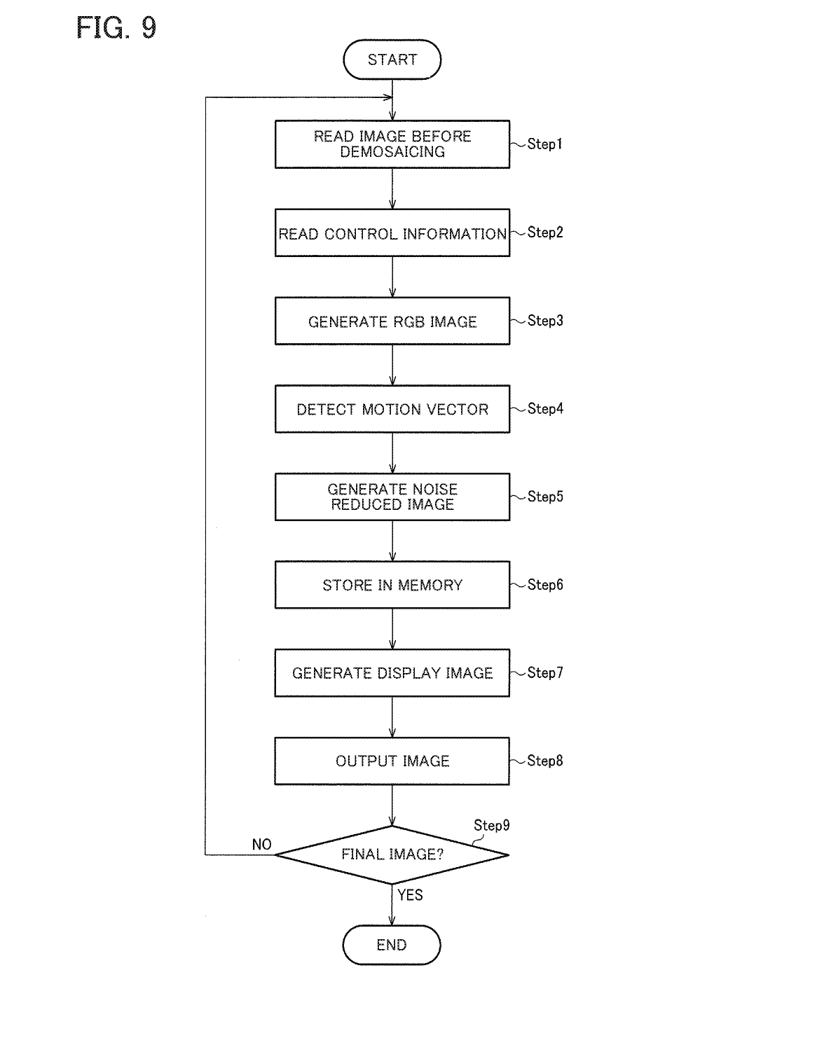

The program is recorded in an information storage medium. The information storage medium may be various recording media, readable by the image processing device, including an optical disk (such as a DVD or a CD), a magneto-optical disk, an HDD, a nonvolatile memory, and a memory such as a random-access memory (RAM). FIG. 9 is a flowchart illustrating a procedure for implementing processes of the interpolation processing section 310, the motion vector detection section 320, the noise reduction section 330, and the display image generation section 350 illustrated in FIG. 1 on an image acquired in advance with software, as an example where a part of the processes performed by the components is implemented by software.

In this case, first of all, an image before demosaicing is read (Step1) and then control information such as various process parameters at the time of acquiring the current image is read (Step2). Next the interpolation process is performed on the image before demosaicing to generate an RGB image (Step3). A motion vector is detected with the method described above by using the RGB image and the recursive RGB image held in the memory described later (Step4). Next, the noise in the RGB image is reduced with the method described above by using the motion vector, the RGB image, and the recursive RGB image (Step5). The RGB image after the noise reduction (NR image) is stored in the memory (Step6) Then, WB or y process or the like is performed on the NR image to generate a display image (Step7). Finally, the display image thus generated is output (Step8). When the series of processes is completed for all the images, the processes are terminated. When there is an unprocessed image, the same processes continue (Step9).

The method according to the present embodiment may be applied to an image processing method (a method for operating an image processing device) including: acquiring an image in time series; obtaining luminance identification information based on a pixel value of the image; and detecting a motion vector based on the image and the luminance identification information, the detecting of the motion vector including setting contribution of a low-frequency component of the image relative to a high-frequency component of the image in the motion vector detection process to be higher with a smaller luminance identified by the luminance identification information.

The image processing device and the like according to the present embodiment may have a specific hardware configuration including a processor and a memory. The processor may be a CPU for example. Note that the processor is not limited to a CPU, and may be various processors such as a graphics processing unit (GPU) or a digital signal processor (DSP). The memory stores a computer-readable command that is executed by the processor so that components of the image processing device and the like according to the present embodiment are implemented. The memory may be a semiconductor memory such as a static RAM or a dynamic RAM, a register, a hard disk, and the like. The command is a command of a command set forming the program.

Alternatively, the processor may be a hardware circuit including an application specific integrated circuit (ASIC). Thus, this processor includes a processor with the components of the image processing device implemented with circuits. Thus, the command stored in the memory may be a command for instructing an operation to the hardware circuit of the processor.

### 1.3 Modification

In the example described above, the luminance signal is used as the luminance identification information. Specifically, in the example described above, the calculation process for the evaluation value and the correction process for the motion vector are switched based on the pixel value Y_LPF_{cur}(x,y) of the low-frequency Y image. The luminance identification information according to the present embodiment may be any information with which a luminance (brightness) of an image can be identified, and thus is not limited to the luminance signal.

For example, a G signal of the RGB image or an R signal or a B signal may be used as the luminance identification information. Alternatively, two or more of the R signal, the G signal, and the B signal may be combined in a method other than that represented by Formula (2) described above, to obtain the luminance identification information.

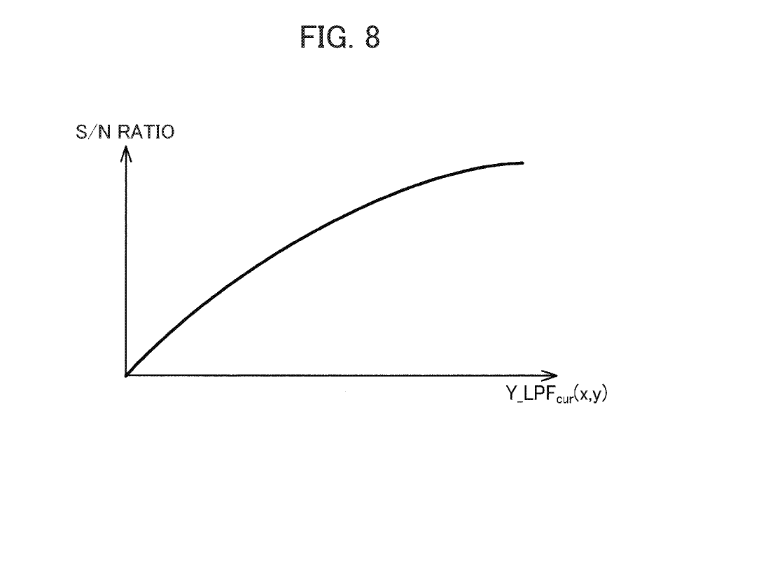

The luminance identification information may be an amount of noise estimated based on the image signal value. Unfortunately, the amount of noise is difficult to directly obtain from an image. Thus, for example, prediction information indicating relationship between the amount of noise and information obtained from an image may be acquired in advance, and the amount of noise may be estimated based on the prediction information. For example, a noise characteristic as illustrated in FIG. 8 may be set in advance. The luminance signal is converted into an amount of noise, and the various coefficients (Coef, Coef′, C) may be controlled based on the amount of noise. This amount of noise is not limited to an absolute value of noise, and a ratio between a signal component and a noise component (an S/N ratio) may be used as illustrated in FIG. 8. The process for a large luminance may be performed when the S/N ratio is high, and the process for a small luminance may be performed when the S/N ratio is low.

In the example described above, the subtraction ratio of the low-frequency image (Y_LPF_{cur},Y_LPFₚᵣₑ) is controlled based on the luminance signal to control the ratio of the low-frequency component in the motion detection image (Y′_{cur},Y′ₚᵣₑ) and the evaluation value. However, this should not be construed in a limiting sense.

For example, a known Laplacian filter or the like may be used on a luminance image to generate a high-frequency image, and the high-frequency image may be added to the luminance image. With an addition ratio of the high-frequency image controlled based on the luminance signal as in the present embodiment, a similar effect can be obtained.

Specifically, the motion vector detection section 320 generates a high-pass frequency image (high-frequency image) by performing a filter process, with a passband at least including a band corresponding to the high-frequency component, on the image, generates the motion detection image by adding the high-pass frequency image to the image at a first addition ratio in a case where the luminance identified by the luminance identification information is small, and generates the motion detection image by adding the high-pass frequency image to the image at a second addition ratio higher than the first addition ratio in a case where the luminance identified by the luminance identification information is large.

Thus, the ratio of the high-frequency component is relatively high in the bright portion and the ratio of the low-frequency component is relatively high in the dark portion. Thus, an effect similar to that obtained with a configuration of subtracting the low-frequency image can be expected.

A spatial frequency component in the high-frequency image can be optimized in accordance with a band of a main target object. For example, in a configuration where the high-frequency image is acquired by proving a bandpass filter to an RGB image, a passband of the bandpass filter is optimized based on the band of the main target object. For a biological image, a spatial frequency corresponding to a fine biological structure (such as capillary blood vessels) is included in a passband of the bandpass filter. Thus, the motion vector can be detected while focusing on the main target object in the bright portion, and thus can be expected to be even more accurately detected.

In the example described above, the motion vector (Vx(x,y),Vy(x,y)) obtained by the motion vector detection section 320 is used for the NR process by the noise reduction section 330. However, the application of the motion vector is not limited to this. For example, stereoscopic images (parallax images) may be used as a plurality of images that are targets of motion vector calculation. In such a case, information about a distance to an object or the like can be obtained by obtaining the parallax based on the magnitude of the motion vector.

Alternatively, in a configuration where the imaging device 200 performs autofocusing, the motion vector may be used as a trigger for a focusing operation for the autofocusing, that is, an operation of searching for a lens position to bring the object into focus by operating the condensing lens 230 (a focus lens in particular). When the focusing operation is performed in a state where the imaging device 200 and an object is in given positional relationship, a state where a desired object is in focus is regarded as being maintained as long as the change in the positional relationship is small. Thus, the focusing operation is less likely to be required to be performed again. In view of this, whether the relative positional relationship between the imaging device 200 and an object has changed may be determined based on a motion vector. Then, the focusing operation may be started when the motion vector exceeds a given threshold, whereby autofocusing can be efficiently performed.

A captured image acquired by a medical endoscope system may include a treatment toll such as a scalpel and forceps. During a medical procedure using an endoscope system, a movement of the treatment tool might result in a large motion vector even in a state where the focusing operation is not required because the positional relationship between the imaging device 200 and a main target object (tissue or lesioned part) is maintained. In such a situation, the local motion vector can be accurately obtained with the method according to the present embodiment. Thus, whether only the treatment tool has moved or the positional relationship between the imaging device 200 and the main target object has also moved can be accurately determined, so that the focusing operation can be performed in an appropriate situation. For example, a level of fluctuation of a plurality of motion vectors obtained from an image may be obtained. A large variation is estimated to indicate a state where movement is different between the treatment tool and the main target object, that is, a state where the treatment tool is moving with the main target object not largely moving.

Thus, the focusing operation is not performed when the variation is large.

### 2. Second Embodiment

### 2.1 System Configuration Example

An endoscope system according to the second embodiment of the present disclosure is described. The image processing section 300 has a configuration that is the same as that in the first embodiment except for the motion vector detection section 320, and thus the description thereof is omitted. In the description below, description on configurations that are the same as those described above will be omitted as appropriate.

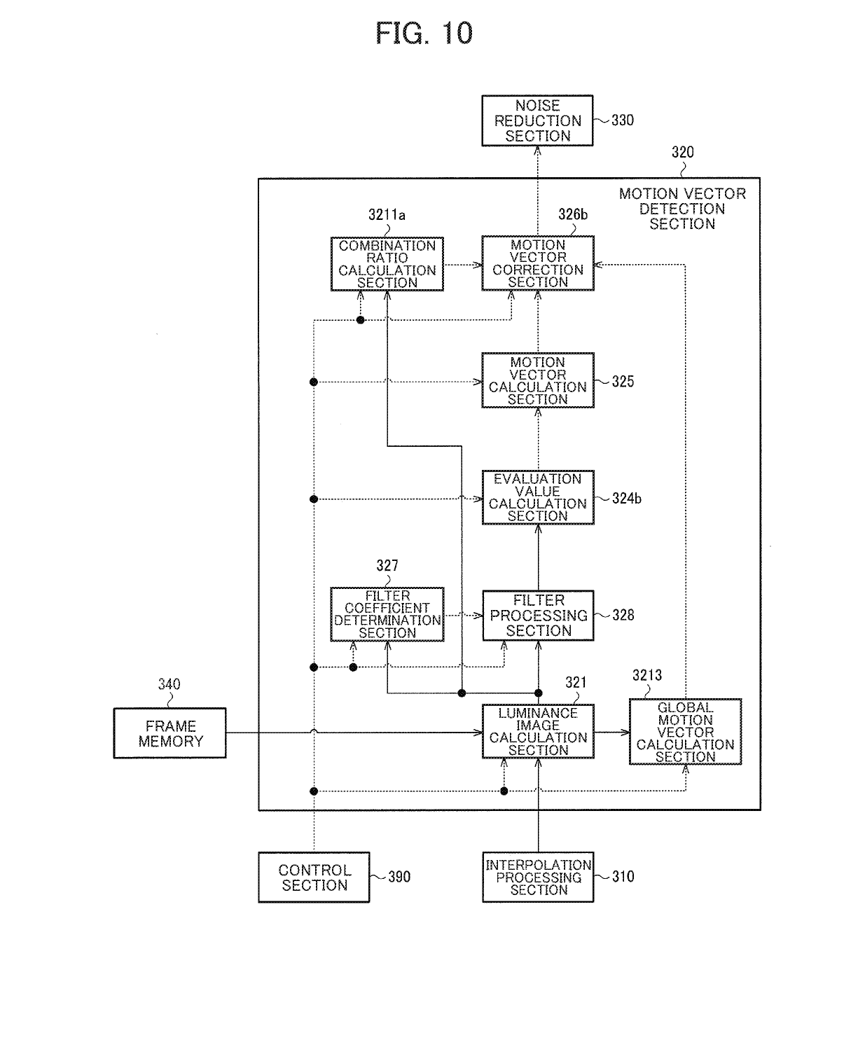

FIG. 10 illustrates the motion vector detection section 320 according to the second embodiment in detail. The motion vector detection section 320 includes the luminance image calculation section 321, a filter coefficient determination section 327, a filter processing section 328, an evaluation value calculation section 324b, the motion vector calculation section 325, the global motion vector calculation section 3213, a motion vector correction section 326b, and a combination ratio calculation section 3211a.

The interpolation processing section 310 is connected to the luminance image calculation section 321. The frame memory 340 is connected to the luminance image calculation section 321. The luminance image calculation section 321 is connected to the filter coefficient determination section 327, the filter processing section 328, and the global motion vector calculation section 3213. The filter coefficient determination section 327 is connected to the filter processing section 328. The filter processing section 328 is connected to the evaluation value calculation section 324b. The evaluation value calculation section 324b is connected to the motion vector calculation section 325. The motion vector calculation section 325 is connected to the motion vector correction section 326b. The motion vector correction section 326b is connected to the noise reduction section 330. The global motion vector calculation section 3213 and the combination ratio calculation section 3211a are connected to the motion vector correction section 326b. The control section 390 is connected to and controls the components of the motion vector detection section 320.

### 2.2 Detail of Motion Vector Detection Process

The luminance image calculation section 321, the global motion vector calculation section 3213, and the motion vector calculation section 325 are the same as those in the first embodiment, and thus detail description thereof will be omitted.

The filter coefficient determination section 327 determines a filter coefficient used by the filter processing section 328 based on Y image Y_{cur}(x,y) output from the luminance image calculation section 321. For example, three types of filter coefficients are switched from one to another based on Y_{cur}(x,y) and given luminance thresholds Y1 and Y2 (Y1<Y2).

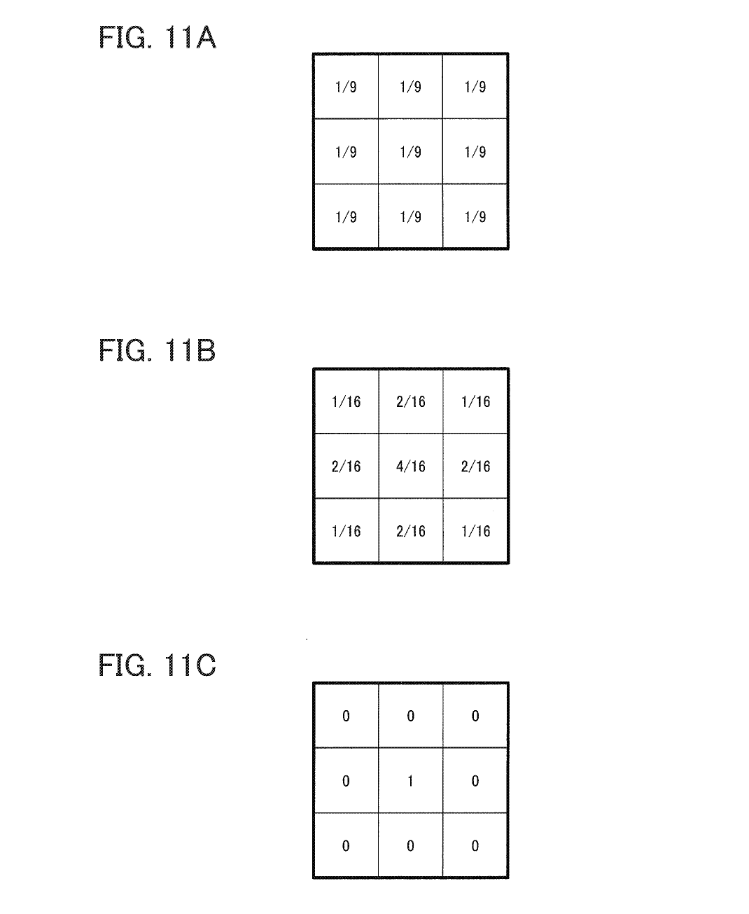

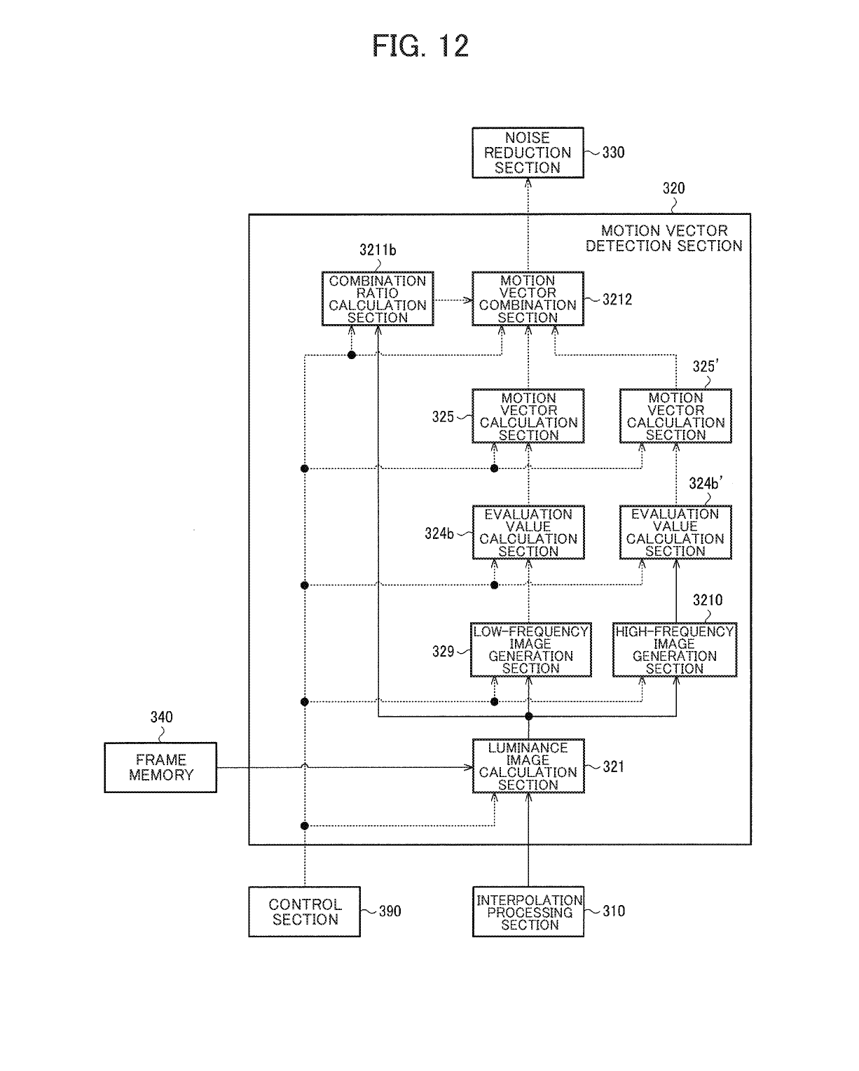

Specifically, a filter A is selected when 0<Y_{cur}(x,y)<Y1 holds true. A filter B is selected when Y1<Y_{cur}(x,y)<Y2 holds true. A filter C is selected when Y2<Y_{cur}(x,y) holds true. The filter A, the filter B, and the filter C are defined in FIG. 11A to FIG. 11C. The filter A is for obtaining a simple average of a process target pixel and peripheral pixels as illustrated in FIG. 11A. The filter B is for obtaining a weighted average of the process target pixel and peripheral pixels as illustrated in FIG. 11B, and involves a higher rate of the process target pixel relative to the filter A. FIG. 11B illustrates an example where the filter B is a Gaussian filter. The filter C is for directly outputting the pixel value of the process target pixel as an output value as illustrated in FIG. 11C.

As illustrated in FIG. 11A to FIG. 11C, the relationship filter A<filter B<filter C holds true in terms of the contribution of the process target pixel to the output value. Thus, the relationship filter A>filter B>filter C holds true in terms of the smoothing level, and thus a filter with a higher level of smoothing is selected for a smaller luminance signal. However, the filter coefficients and the switching method are not limited to these. Y1 and Y2 may each be set to be a predetermined value, or may be set by the user through the external I/F section 500.

The filter processing section 328 uses the filter coefficient determined by the filter coefficient determination section 327 to perform a smoothing process on the Y image and the recursive Y image calculated by the luminance image calculation section 321 to acquire a smoothed Y image and a smoothed recursive Y image.

The evaluation value calculation section 324b uses the smoothed Y image and the smoothed recursive Y image to calculate an evaluation value. This calculation is performed with a sum of absolute differences (SAD) or the like widely used in block matching.

The motion vector correction section 326b performs a correction process on the motion vector (Vx′(x,y),Vy′(x,y)) calculated by the motion vector calculation section 325. Specifically, the motion vector (Vx′(x,y),Vy′(x,y)) and the global motion vector (Gx,Gy) calculated by the global motion vector calculation section 3213 are combined to obtain a final motion vector (Vx(x,y),Vy(x,y)) as in the following Formula (8).

Vx(x,y)={1−MixCoefV(x,y)}×Gx+MixCoefV(x,y)×Vx′(x,y)

Vy(x,y)={1−MixCoefV(x,y)}×Gy+MixCoefV(x,y)×Vy′(x,y) (8)

The combination ratio calculation section 3211a calculates MixCoefV(x,y). Specifically, the combination ratio calculation section 3211a calculates the combination ratio MixCoefV(x,y) based on the luminance signal output from the luminance image calculation section 321. The combination ratio is characterized in that it increases in accordance with the luminance signal, and may have a characteristic similar to that of Coef(x,y) described above with reference to FIG. 5A and FIG. 5B for example.

In this example, MixCoefV and 1−MixCoef respectively represent combination rates of the motion vector (Vx′(x,y),Vy′(x,y)) and the global motion vector (Gx,Gy). Thus, Formula (8) described above is equivalent to Formula (7) described above. Note that the combination rates are not limited to those in Formula (8) described above, and may be any values as long as the combination rate of the motion vector (Vx′(x,y),Vy′(x,y)) decreases as the luminance decreases.

The motion vector detection section 320 according to the present embodiment generates the motion detection image by performing a first filter process with a first smoothing level on the image in a case where the luminance identified by the luminance identification information is small, and generates the motion detection image by performing a second filter process with a lower smoothing level than the first filter process on the image in a case where the luminance identified by the luminance identification information is large.

The number of filters with different smoothing levels can be modified in various ways. A larger number of filters enables the rate of the low-frequency component in the motion detection image to be controlled more in detail. However, a larger number of filters can also be disadvantageous. Thus, the number of filters may be specifically determined based on the allowable circuit size, process time, memory capacity and the like.

The smoothing level is determined in accordance with the contribution of the process target pixel and peripheral pixels. For example, the smoothing level may be controlled by adjusting the coefficient (rate) applied to each pixel as illustrated in FIG. 11A to FIG. 11C. The filter size is not limited to those of 3×3 filters illustrated in FIG. 11A to FIG. 11C, and may be changed to control the smoothing level. For example, an averaging filter for obtaining a simple average can have a larger size to provide a higher smoothing level.

With the method described above, the intense smoothing process is provided to the dark portion with a large amount of noise, so that the motion vector is detected with noise sufficiently reduced, whereby erroneous detection due to noise can be suppressed. A less intense smoothing process or no smoothing process is provided to the bright portion with a small amount of noise, whereby degradation of the detection accuracy for the motion vector can be suppressed.

Furthermore, a coefficient of determination of the reference vector (global motion vector) is set to be large as illustrated in Formula (8) described above for the dark portion with a large amount of noise. Thus, variation of the motion vector due to erroneous detection can be suppressed, whereby an effect of suppressing a motion (artifact) that is not actually made by an object can be obtained. The reference vector may be a vector (a zero vector for example) other than the global motion vector, as in the first embodiment.

### 2.3 Modification

In the present embodiment, the motion detection image used in the evaluation value calculation is generated through the smoothing process. However, this should not be construed in a limiting sense. For example, the evaluation value may be detected by using a composite image obtained by combining a high-frequency image generated with an appropriate bandpass filter with a smoothed image (low-frequency image) generated by the smoothing process. When the luminance signal is small, the combination rate of the low-frequency image is set to be large so that higher noise resistance can be achieved.

The motion vector can be expected to be more accurately detected by optimizing the band of the bandpass filter for generating the high-frequency image in accordance with the band of the main target object, as in the modification of the first embodiment.

The processes performed by the image processing section 300 according to the present embodiment may be partially or entirely implemented with software, as in the first embodiment.

### 3. Third Embodiment

### 3.1 System Configuration Example

An endoscope system according to a third embodiment of the present disclosure is described. The image processing section 300 has a configuration that is the same as that in the first embodiment except for the motion vector detection section 320, and thus the description thereof is omitted.

FIG. 12 illustrates the motion vector detection section 320 according to the third embodiment in detail. The motion vector detection section 320 includes the luminance image calculation section 321, the low-frequency image generation section 329, a high-frequency image generation section 3210, two evaluation value calculation sections 324b and 324b′ (performing the same operation), two motion vector calculation sections 325 and 325′ (performing the same operation), a combination ratio calculation section 3211b, and a motion vector combination section 3212.

The interpolation processing section 310 and the frame memory 340 are connected to the luminance image calculation section 321. The luminance image calculation section 321 is connected to the low-frequency image generation section 329, the high-frequency image generation section 3210, and the combination ratio calculation section 3211b. The low-frequency image generation section 329 is connected to the evaluation value calculation section 324b. The evaluation value calculation section 324b is connected to the motion vector calculation section 325. The high-frequency image generation section 3210 is connected to the evaluation value calculation section 324b′. The evaluation value calculation section 324b′ is connected to the motion vector calculation section 325′. The motion vector calculation section 325, the motion vector calculation section 325′, and the combination ratio calculation section 3211b are connected to the motion vector combination section 3212. The motion vector combination section 3212 is connected to the noise reduction section 330. The control section 390 is connected to and controls the components of the motion vector detection section 320.

### 3.2 Detail of Motion Vector Detection Process

The low-frequency image generation section 329 performs a smoothing process on a luminance image by using a Gaussian filter (FIG. 11B) for example, and outputs the low-frequency image thus generated to the evaluation value calculation section 324b.

The high-frequency image generation section 3210 extracts high-frequency component from the luminance image by using a Laplacian filter and the like for example, and outputs the high-frequency image thus generated to the evaluation value calculation section 324b′.

The evaluation value calculation section 324b calculates an evaluation value based on the low-frequency image, and the evaluation value calculation section 324b′ calculates an evaluation value based on the high-frequency image. The motion vector calculation sections 325 and 325′ calculates motion vectors from respective evaluation values output from the evaluation value calculation sections 324b and 324b′.

The motion vector calculated by the motion vector calculation section 325 is defined as (VxL(x,y),VyL(x,y)), and the motion vector calculated by the motion vector calculation section 325′ is defined as (VxH(x,y),VyH(x,y)). The motion vector (VxL(x,y),VyL(x,y)) corresponds to the low-frequency component, and the motion vector (VxH(x,y),VyH(x,y)) corresponds to the high-frequency component.

The combination ratio calculation section 3211b calculates the combination ratio MixCoef(x,y) of the motion vector calculated based on the low-frequency image, based on the luminance signal output from the luminance image calculation section 321. The combination ratio is characterized in that it increases in accordance with the luminance signal, and may have a characteristic similar to that of Coef(x,y) described above with reference to FIG. 5A and FIG. 5B for example.

The motion vector combination section 3212 combines the two types of motion vectors based on the combination ratio MixCoef(x,y). Specifically, the motion vector (Vx(x,y),Vy(x,y)) is obtained with the following Formula (9).

Vx(x,y)={1−MixCoef(x,y)}×VxL(x,y)+MixCoef(x,y)×VxH(x,y)

Vy(x,y)={1−MixCoef(x,y)}×VyL(x,y)+MixCoef(x,y)×VyH(x,y) (9)

The motion vector detection section 320 according to the present embodiment generates a plurality of motion detection images with different frequency components based on the images, and detects the motion vector by combining the plurality of motion vectors detected from the respective plurality of motion detection images. The motion vector detection section 320 sets the combination rate of the motion vector detected from the motion detection image (low-frequency image) corresponding to the low-frequency component to be relatively larger with a smaller luminance identified by the luminance identification information.

With the method described above, the motion vector calculated based on the low-frequency image with the influence of noise reduced is dominant in the dark portion with a large amount of noise, whereby erroneous detection can be suppressed. On the other hand, the motion vector calculated based on the high-frequency image enabling the motion vector to be highly accurately detected is dominant in the bright portion with a small amount of noise, whereby high-performance motion vector detection is implemented.

What is claimed is:

1. An image processing device comprising a processor including hardware,
the processor performing:
an image acquisition process of acquiring an image in time series; and
a motion vector detection process including obtaining luminance identification information based on a pixel value of the image, and detecting a motion vector based on the image and the luminance identification information,
the processor
setting contribution in the motion vector detection process to be higher with a smaller luminance identified by the luminance identification information, the contribution being contribution of a low-frequency component of the image relative to a high-frequency component of the image.

2. The image processing device as defined in claim 1, wherein
the processor
generates a motion detection image used for the motion vector detection process based on the image, and
sets a rate of the low-frequency component in the motion detection image to be higher in a case where the luminance identified by the luminance identification information is small than in a case where the luminance is large.

3. The image processing device as defined in claim 2, wherein
the processor
generates the motion detection image by performing a first filter process with a first smoothing level on the image in a case where the luminance identified by the luminance identification information is small, and
generates the motion detection image by performing a second filter process with a lower smoothing level than the first filter process on the image in a case where the luminance identified by the luminance identification information is large.

4. The image processing device as defined in claim 2, wherein
the processor
generates a smoothed image by performing a predetermined smoothing filter process on the image,
generates the motion detection image by subtracting the smoothed image from the image at a first subtraction ratio in a case where the luminance identified by the luminance identification information is small, and
generates the motion detection image by subtracting the smoothed image from the image at a second subtraction ratio higher than the first subtraction ratio in a case where the luminance identified by the luminance identification information is large.

5. The image processing device as defined in claim 2, wherein
the processor
generates a high-pass frequency image by performing a filter process, with a passband at least including a band corresponding to the high-frequency component, on the image,
generates the motion detection image by adding the high-pass frequency image to the image at a first addition ratio in a case where the luminance identified by the luminance identification information is small, and
generates the motion detection image by adding the high-pass frequency image to the image at a second addition ratio higher than the first addition ratio in a case where the luminance identified by the luminance identification information is large.

6. The image processing device as defined in claim 1, wherein
the processor
calculates a difference between a plurality of the images acquired in time series as an evaluation value, detects the motion vector based on the evaluation value, and
sets the contribution of the low-frequency component of the image relative to the high-frequency component of the image in the calculation process for the evaluation value to be higher with a smaller luminance identified by the luminance identification information.

7. The image processing device as defined in claim 6, wherein
the processor
corrects, in the motion vector detection process, the evaluation value to facilitate detection of a given reference vector.

8. The image processing device as defined in claim 7, wherein
the processor
corrects the evaluation value so that the detection of the reference vector is further facilitated with a smaller luminance identified by the luminance identification information.

9. The image processing device as defined in claim 6, wherein
the processor
performs a correction process on the motion vector obtained based on the evaluation value, the correction process being performed based on the luminance identification information so that the motion vector becomes close to a given reference vector.

10. The image processing device as defined in claim 9, wherein
the processor
performs the correction process so that the motion vector becomes closer to the given reference vector with a smaller luminance identified by the luminance identification information.

11. The image processing device as defined in claim 7, wherein
the reference vector is
a global motion vector representing a global motion as compared with the motion vector detected based on the evaluation value or is a zero vector.

12. The image processing device as defined in claim 1, wherein
the processor
generates a plurality of motion detection images with different frequency components based on the image, detects the motion vector by combining a plurality of motion vectors detected from a plurality of the respective motion detection images, and
sets a combination rate of the motion vector detected from the motion detection image corresponding to the low-frequency component to be relatively larger with a smaller luminance identified by the luminance identification information.

13. An endoscope system comprising: an imaging device that acquires an image in time series; and
a processor including hardware,
the processor performing:
a motion vector detection process including obtaining luminance identification information based on a pixel value of the image, and detecting a motion vector based on the image and the luminance identification information,
the processor setting contribution in the motion vector detection process to be higher with a smaller luminance identified by the luminance identification information, the contribution being contribution of a low-frequency component of the image relative to a high-frequency component of the image.

14. An information storage device storing a program,
the program causing a computer to perform the steps of
acquiring an image in time series,
obtaining luminance identification information based on a pixel value of the image, and
detecting a motion vector based on the image and the luminance identification information,
the detecting of the motion vector including
setting contribution in the motion vector detection process to be higher with a smaller luminance identified by the luminance identification information, the contribution being contribution of a low-frequency component of the image relative to a high-frequency component of the image.

15. An image processing method comprising: acquiring an image in time series;
obtaining luminance identification information based on a pixel value of the image; and
detecting a motion vector based on the image and the luminance identification information,
the detecting of the motion vector including
setting contribution in the motion vector detection process to be higher with a smaller luminance identified by the luminance identification information, the contribution being contribution of a low-frequency component of the image relative to a high-frequency component of the image.
